(19) United States
(12) Patent Application Publication
(10) Pub. No.: US 2004/0138558 A1
(43) Pub. Date: Jul. 15, 2004

(54) Methods and devices for detecting tissue cells

(76) Inventors: Robert J. Dunki-Jacobs, Mason, OH (US); Yoav Avidor, Cincinnati, OH (US); Michael P. Weir, Blanchester, OH (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/713,637

(22) Filed: Nov. 14, 2003

Related U.S. Application Data
(60) Provisional application No. 60/426,211, filed on Nov. 14, 2002.

(51) Int. Cl. A61B 6/00

(52) U.S. Cl. 600/431; 600/436; 424/9.34

(57) ABSTRACT

Devices and methods are provided for identifying tissue cells, such as cancerous cells. The device can include a swallowable capsule having a detector. A patient can be given a substance which includes a marker material (such as a radioactive marker or a magnetic marker material), and which substance can be preferentially bound to or otherwise associated with the particular cell type.

This patent application claims priority to U.S. Provisional Application 60/426,211 filed Nov. 14, 2002.

This patent application cross references and incorporates by reference U.S. Patent Application “Methods and Devices For Detecting Abnormal Tissue Cells”, docket number END 5005NP filed on the date of filing this application.

### FIELD OF THE INVENTION

The present invention is related generally to medical devices and methods, and more particularly to devices and methods for detecting tissue types, including abnormal tissue cells, such as cancerous tissue cells.

### BACKGROUND OF THE INVENTION

Colorectal cancer is the third most common cancer in the United States, and the second in terms of annual cancer mortality. Each year, over 130,000 Americans are diagnosed with this disease. Fortunately, unlike many other cancers the prognosis associated with a diagnosis of colorectal cancer can be optimistic if the cancer is discovered early. When discovered at an early stage, the 5-year survival and cure rate can be over 90%. Hence the value of general screening for colorectal cancer, which is recommended in the United States for every adult over 50 years-of age.

Current screening modalities for colorectal cancer include occult fecal blood (Hemoccult), barium enema, sigmoidoscopy, colonoscopy, and experimental technologies such as CT Virtual Colonography and fecal DNA testing. These modalities can detect some small and early cancers. However, like any diagnostic modality, their adoption as a mass screening tool depends on their ability to provide benefits such as low cost testing, reliable sensitivity in detecting malignancy, and good specificity as to indicating the location of the malignancy in the patient's body.

Fecal occult blood screening can be easy to administer and relatively low cost, but is sometimes also associated with low sensitivity for cancer. Additionally, patients may find repeated retrieval of specimens from fresh stool objectionable and demeaning.

Sigmoidoscopy can provide higher sensitivity for disease in the left (descending) colon. Accuracy of sigmoidoscopy may be sensitive to physician expertise. Additionally, patients may find the total colon cleansing regimen (“bowel prep”) and pre-procedure dietary restrictions objectionable.

Colonoscopy provides relatively high sensitivity and specificity. However, colonoscopy can require advanced physician expertise that increases costs and limits its use in a mass-scale setting. The additional cost associated with the administration of conscious sedation may also limit adoption of this procedure as a screening methodology. As with sigmoidoscopy, patients may find the total colon cleansing regimen (“bowel prep”) and pre-procedure dietary restrictions objectionable.

Virtual colonoscopy based on 3D Computed Tomography or Magnetic Resonance image sets is currently under development. While the sensitivity and specificity of this approach is still being debated, either imaging modality would require a bowel prep and colon insufflation (an uncomfortable part of the sigmoidoscopy and colonoscopy procedure) in order to achieve acceptable results.

Fecal DNA testing may provide more sensitivity than fecal occult blood testing. However, the specimen collection mechanism can be substantially the same as that for fecal occult blood and therefore patients may find retrieval of specimens from fresh stool objectionable.

The literature discloses capsules for use in the GI tract. Pluzhnikov et al (U.S. Pat. No. 3,690,309) discloses a radiation-detecting capsule with a particular configuration of circuitry designed to minimize power consumption. Hassan and Pearce, in Phys Med Biol, 1978, vol 23, no. 2, describe a radiation-detecting capsule using a particular detector and continuous analog transmission of the detected signal. Lambert et al, in Medical and Biological Engineering and Computing, March 1991, describe a versatile, multifunction capsule with mechanical position tracking and material sampling capabilities. Glukhovsky, in European Patent Application EP 1 159 917 (2001), describes a capsule with capabilities for multiple electrical impedance measurement for distinguishing tissue variation. Kimchy et al (U.S. application Ser. No. 2002/0099310) describes a capsule-based approach for use in the Gastro Intestinal Tract.

Additionally, Goldberg (U.S. Pat. No. 5,716,595) and Lemelson (U.S. Pat. No. 5,993,378) describe the use of substances such as monoclonal antibodies and antibody fragments having biological affinity for a tissue type.

Still, scientists continue to seek improved methods for use in detection of abnormal tissue in the Gastro Intestinal Tract.

### SUMMARY OF THE INVENTION

Applicants have recognized a number of unmet needs in connection with devices and methods for use in detecting tissue types in the Gastro Intestinal Tract, including the need to manage the data received or generated by a detection system, analyze and present the data in a form suitable for large numbers of cases in an efficient way; the challenge of dealing with large amounts of the differentiating and marking material which will often remain in circulation or untargeted tissue, in comparison with the small amount actually bound to the suspect or targeted tissue; the need to provide effective control of power consumption in the capsule prior to its application.

In one embodiment, the present invention provides a swallowable capsule comprising: a detector; a pulse shaping device; and at least one single channel analyzer. In another embodiment, the present invention provides a method for detecting target cells in a patient comprising: marking target cells in the patient with a substance capable of being detected; directing a detector through a naturally occurring body lumen in the patient to detect signals from the substance; and mathematically transforming data representing at least some of the signals detected. Signals detected can be grouped by energy level to provide a histogram or other graphical representation of the number of counts received in discrete energy ranges. The signals detected can be compared with a predetermined model or pattern of response to determine the probability that a tumor or other target tissue is being detected when a characteristic response is received.

### BRIEF DESCRIPTION OF THE DRAWINGS

The novel features of the invention are set forth with particularity in the appended claims. The invention itself, however, both as to organization and methods of operation, together with further objects and advantages thereof, may best be understood by reference to the following description, taken in conjunction with the accompanying drawings in which:

FIG. 1 is a schematic illustration of a test system according to one embodiment of the present invention showing the various component parts of the system.

FIG. 2 is a schematic illustration of a detection capsule according to one embodiment of the present invention.

FIG. 3 is a block diagram schematic illustration of a detection capsule in a radiation detection embodiment of the present invention.

FIG. 4 is a block diagram schematic illustration of a detection capsule in a magnetic particle detection embodiment of the present invention.

FIG. 5 is a block diagram schematic illustration of a patient data collection unit according to one embodiment of the present invention.

FIG. 6 is a schematic illustration showing a detection capsule and associated protective packaging according to one embodiment of the present invention.

FIG. 7 is a schematic illustration of one embodiment of a Physician Workstation according to one embodiment of the present invention.

FIG. 8 is a schematic illustration of a graphical report that can be generated according to one embodiment of the present invention.

FIG. 9 is a schematic illustration showing relative performance of several detector schemes.

FIG. 10 illustrates the detector response of a typical Scintillation Detection (SD) radiation detector.

### DETAILED DESCRIPTION OF THE INVENTION

The present invention provides medical devices and methods for detecting abnormal tissue, such as cancerous tissue. The invention is especially applicable for use in detecting cancer of the gastrointestinal tract, such as colon, rectal, gastric, esophageal, small bowel cancer and lymphoma, as well as adjacent organ disease like pancreatic cancer. While the present invention describes use for cancer, it could also be used for benign diseases such as Chrohn's disease. While the present invention is described with respect to use with a human patient, it will be understood that the present invention is applicable for use with non-human patients.

Detection Method/Radiation Method

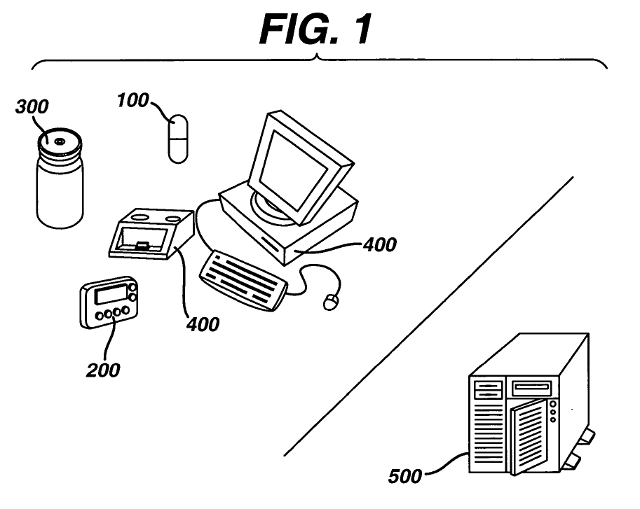

In one embodiment, the present invention provides a method for locating abnormal tissue growth, such as cancer. Referring to FIG. 1, the method can include providing a substance having an affinity for a target tissue type, such as cancer, and a capability for providing a detectable signal, such as the substance 300 (which can be in the form of an injectable liquid in a vial); administering the substance 300 to the patient; providing an swallowable pill or capsule, such as the detector capsule 100 having a detector for receiving a signal emitted by the substance; directing the capsule with detector through at least a portion of the patient's gastrointestinal tract (GIT); communicating the received signals to a data collection device, such as the patient data collection unit (PDCU) 200 having a data communication link with the detector capsule and a means for storage of said data; analyzing the data, such as with a data collection and analysis center (DCAC) 500 having a means to gather said data from a plurality of PDCUs and to organize said data into human readable form; and providing a human interface for management of the method and display of said human readable form of the data, such as the physicians workstation 400 enabling a skilled observer to determine the presence and location of cancerous material.

By giving the patient a substance that has a relatively high affinity to the cancer cells, and that also emit a certain signal, the observer can note if and where the signal is coming from. It is useful to use the terms “differentiation” or “differentiator” for the tissue-selective interaction, and “marking” or “marker” for the provision of some detectable aspect; however, the “mark” or “marker” terminology is often employed to encompass both functions.

A suitable differentiator is useful in identifying a certain cell type, such as a cancerous cell, but does not single out “innocent bystander” cells that are normal. Examples of such a differentiating material are the “tumor associated antigens”. This name makes the point that this antigen (protein) is associated only or at least overwhelmingly with cancer cells, while it is substantially absent from normal cells.

In radiolabeled radiation imaging systems, such as a gamma camera or SPECT imager, a collimator can be used to provide a directional response, such that particles emanating from a constrained physical region (2-dimensional: “pixel”; 3-dimensional “voxel”) of the object being imaged can be distinguished from other such regions. Typically such a device intercepts thousands of particles and relates them to an associated pixel (Gamma Camera) or voxel (SPECT imager). Note that in discussing the products of nuclear decay, a distinction between particles and “rays” may sometimes be found, though the terms ray and particle are used interchangeably in this discussion.

The sensitivity of the detector and the ability to spatially resolve the distribution of the radiation sources can be constrained by the distance between the detector and sources and by the intervening material. In free space, since the sources are composed of isotropic radiators, the flux as seen at a detector varies inversely with the square of the distance to the source. In the body, the radiation is both absorbed and scattered. As it is scattered, its direction is changed and its energy reduced. The result is that the reduction with distance is even more severe than inverse square. An external detector is inevitably challenged to acquire a good “picture” of the distribution of radiation in the patient because of the high attenuation and loss of directionality. A further difficulty experienced with external detectors is the partial volume effect, where a point source's radiation is observed in multiple (4 for 2d and 8 for 3D) pixels or voxels at attenuations of up to 75% or 88%). An internal detector, as described in this invention, possesses a detection advantage.

Detection Method/Magnetic Method

In an alternative embodiment to radiation detection, the equipment and materials are similar to those just described, except that the substance 300 provided in the marker vial does not incorporate a radioactive (self-emitting) material. Instead, it incorporates a material that, in response to an activating or probe signal, creates a response that is detected by the capsule 100. The response is conveyed to the data collection device 200 and processed as previously described. Examples of such a substance includes a magnetic material, such as in the form of magnetizable particles. The magnetic substance, when subjected to a magnetizing field, result in a detectable distortion of the field, or a temporal signature of magnetization or demagnetization, which is detectable. Such an embodiment avoids the use of radioactive substances. It has the further advantages that magnetic fields, specifically dipole and higher moment ones, can exhibit a faster reduction with distance than the simple radiation model. This facilitates discrimination between the signal from targeted tissue and that from coincidental distributions elsewhere in the body, but farther away.

Devices

Materials for Binding and Marking

Substances 300 useful in the present invention include a signal emitting material (a “marker”) such as a radioactive material, magnetic material, fluorescent material, or ultrasonic contrasting agent in combination with one or more materials that bind preferably to cancer cells, while normal tissue is substantially not bound (a “differentiator”). In one embodiment, a suitable substance can comprise one or more radioactive markers in combination with a protein or protein complex differentiator that has an affinity for a particular target cell type.

A suitable marker can comprise one or more radioactive nuclides. Radioactive nuclides useful in the present invention include those that emit gamma radiation and whose stable isotope is biologically acceptable. In some applications it can be desirable for a radioactive marker to have a half-life comparable to or longer than the nominal transit time of ingested material through the subject gastrointestinal system. It can also be desirable to use an entity that emits gamma radiation above the ambient background (about 100 keV) and low enough to be efficiently collected in detection devices (less than about 1 MeV). Suitable radioactive isotopes include but are not limited to ⁴⁸Cr, ^{99m}Tc, ⁶⁴Cu, ¹⁵³Dy, ¹⁵⁵Dy, ¹⁵⁷Dy, ¹⁸⁸Ir, ⁵²Fe, ³⁸K, ⁸³Sr, ¹²²Xe, ¹²⁵Xe, ⁸⁷Y, ⁶⁶Ga, ²⁰¹Tl, ¹¹¹In, and ¹⁰⁹In. In one embodiment the marker is ^{99m}Tc, the metastable isotope of the element Technetium, which decays by emitting a single gamma particle at 143 keV with a half-life of 6.01 hours.

A suitable differentiator can be one or more monoclonal antibodies (MAb). Monoclonal antibodies useful in the present invention include, but are not limited to those that have an affinity for the TAG-72 protein such as the commercial product Oncoscint® (Cytogen Corporation), the carcinoembryonic antigen (CEA) such as the commercial product CEAscan® (Immunomedics®, Inc.) or other proteins associated with colorectal cancer such as 17-1A.

The following are incorporated herein by reference in their entirety: “Clinical and Technical Considerations for Imaging Colorectal Cancers with Technetium-99m-labeled AntiCEA Fab Fragment” by Deborah A. Erb and Hani A. Nabi of Dept of Nuclear Medicine, SUNY at Buffalo N.Y., Journal of Nuclear Medicine Technology, Volume 28, Number 1, March 2000; “Indium-111 Satumomab Pendetide: The first FDA Approved Monoclonal Antibody for Tumor Imaging” by Paul J. Bohdiewicz, Nuclear Medicine Dept. William Beaumont Hospital, Royal Oak, Mich., Journal of Nuclear Medicine Technology, Volume 26, Number 3, September 1998.

In an alternative embodiment, the differentiator can be selected from a group including peptides and nucleotides.

Where a detection capsule incorporating magnetic detector is employed, the marker can comprise a magnetic or magnetizable nanoparticle. Such particles might be made of Fe₃O₄, gamma-Fe₂O₃, cobalt, and other materials that are conjugated to a MAb, peptides or nucleotides in a similar fashion to the previously described radioactive marker.

In an alternative embodiment, other materials can be used in addition to or in place of the monoclonal antibodies for carrying or otherwise directing a substance to targeted cells or organs. For instance, a material comprising an aqueous core and one or more outer layers (including lipid containing layers such as phospholipid layers) can be used for conveying the marking material to a target cell or organ. A suitable substance includes one or more liposomes. The term liposome, as used herein, refers to an artificial microscopic vesicle having an aqueous core enclosed in one or more phospholipid layers, used to convey a substance such as vaccines, drugs, radioactive materials, enzymes, or other substances to target cells or organs. Suitable commercially available liposomes include Abelcet®, which is Amphotericin B, manufactured by The Liposome Company, Inc., One Research Way, Princeton, N.J. 08540-6619, and Doxil®, which is Doxorubicin, manufactured by ALZA Corporation, 1900 Charleston Rd., Mountain View, Calif. 94039-7210. See also Harrington, Mohammadtaghi et al, “Effective targeting of solid tumors in patients with locally advanced cancers by radiolabeled pegylated liposomes,” Clinical Cancer Research 7, Feb. 2001, incorporated herein by reference.

According to one embodiment of the present invention employing a radiation detector, the substance 300 can include a material comprising, in combination, a differentiator such as an MAb and a marker such as ^{99m}Tc.

Capsule

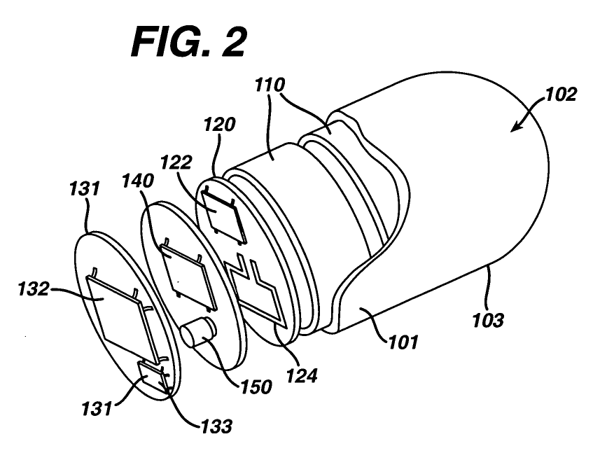

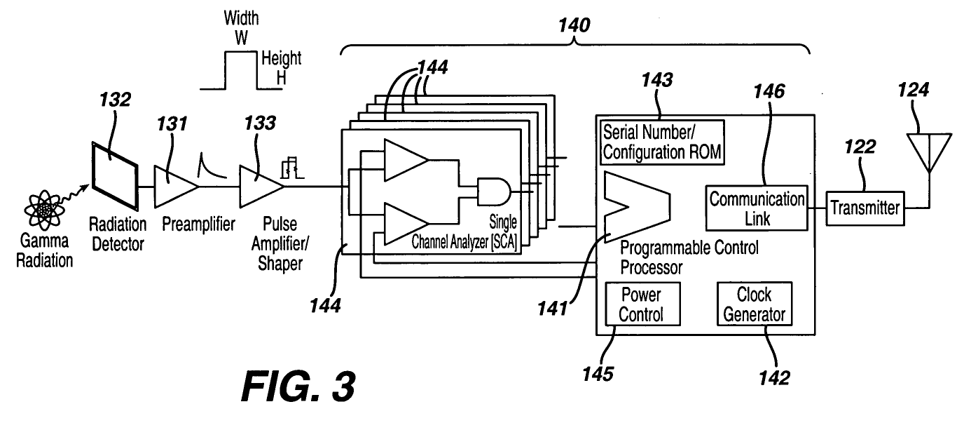

Referring to FIGS. 2 and 3, a capsule 100 adapted for swallowing by the patient can be provided with a detector 132, which can be mounted on or otherwise be a part of a detector module 130 supported in the capsule 100. The detector 132 is capable of detecting the signal emitted by the marker material. Because the marker is associated selectively with cancerous cells (or other target tissue cells) via the differentiator substance, the locally dense concentration of the differentiator in cancerous tissue cells will be detected by the detector onboard the capsule as it passes in close proximity to the cancerous tissue.

Upon ingestion, the capsule travels through the gastrointestinal tract, such as by normal peristalsis. The signal may be transmitted by the capsule immediately to a receiver or to a patient data collection unit (PDCU) 200, which may be positioned outside or inside the body, or recorded in the capsule for later interpretation. For instance, the PDCU can comprise a device that can be supported on the patient's wrist, fastened at the patient's waist, or otherwise associated with the patient's body or clothing during the time the capsule 100 is passing through the GIT (gastro-intestinal tract). The capsule 100 is later excreted in the stool in the normal fashion, and can be retrieved if necessary.

The capsule 100 can comprise any detector 132 suitable for detecting the presence of the marker substance administered to the patient. Suitable detectors include but are not limited to ionizing radiation detectors or magnetic particle detectors. Ionizing radiation detectors can be based on solid-state direct radiation detectors or photo-detectors with attached scintillation crystals. Magnetic particle detectors can be based on sensitive magnetometers, reluctance meters, or temporal response to an applied magnetizing field. Alternatively, a detector module can be located on a flexible endoscope, such as on a colonoscope or a sigmoidoscope.

The capsule 100 can also include one or more power source, such as one or more battery modules 110. Alternatively, the capsule 100 can receive power via a radio frequency (RF) power source; The capsule can also include a transmitter 122 associated with a transmission module 120 for sending raw or processed signal data received by the detector to the receiver 201 or other remote location outside the patient's body, and/or a recorder for recording the signal received by the detector. The receiver 201 outside the patient's body can be adapted to receive and/or record the signal sent from the capsule. Capsule 100 can have an outer surface 101 shaped to aid in ingesting the capsule, and can include one or more coatings 103, one of which can be a protective coating that is acid tolerant. Other organic and inorganic coatings can be applied. By example, coating the surface with Manganese dioxide (MnO₂) may create a laxative effect resulting in more rapid passage of the capsule through the tract. Coating the surface with a diuretic such as loop diuretics (e.g. bumetanides, furosemide), thiazide diuretics (e.g. hydrochlorothizide, chlorozide and chloralidone) and potassium sparing diuretics (e.g. amiloridetramterene) may be helpful in causing accelerated elimination of unassociated markers in the kidney and urinary tract. Alternately, the desired biological effects listed above can be obtained in the normal fashion (i.e. by oral methods) rather than as a coating on the capsule 100.

The capsule 100 can have a generally hemispherically shaped end cap 102, though other smooth tapered shapes can also be employed. Only one generally hemispherically shaped cap is shown in FIG. 2, though it will be understood that such a shaped end cap 102 can be disposed on one or both ends of the capsule 100.

The capsule 100 can include one or more battery modules 110 for providing onboard power or energy. The capsule can also include a transmission module 120 including a RF antenna 124 and a RF transmission circuit 122, plus support, control and logic circuits, powered by the onboard battery. In one embodiment, the transmission module components 122 and 124 comprise an active RF transmitter, meaning that the communication function is achieved by supplying radiating energy from an onboard power source. In an alternative embodiment the transmission module components 122 and 124 comprises a passive or “zero-power” RF transmitter, meaning that the communication function is achieved by altering the apparent RF load seen by a remote RF transmitting power source. In this embodiment, the remote RF power source can also provide a portion of or all of the onboard power requirements reducing or eliminating the need for energy supplied by battery modules 110. One suitable battery chemistry is silver oxide as represented by the Duracell D357 coin cell battery.

The transmission module 120 is selected for efficient short-range unlicensed operation. Low-power implementations of the transmitters 122 incorporated in the Bluetooth® or IEEE 802.11b standards provided, for example, in the Agilent Technologies E8874A Wireless LAN Design Library that can be incorporated into a single purpose radio frequency integrated circuit or as part of an Application Specific Integrated Circuit (ASIC) are suitable. If desired, a custom protocol optimized for low data rate communication and reduced energy usage can be used.

Referring now to FIG. 3, a programmable control processor 141 can be based on a common commercial microcontroller core such as one based on the Intel 8051 8-bit processor instruction set and architecture. Instructions governing the operation of the capsule can be stored in a read-only memory embedded in the microcontroller core module. The microcontroller core can also be responsible for the management, control, and data transfer between all portions of the ASIC and attached components.

A clock generation and timing module 142 can be used for generation of all internal clock and timing signals. A write-once configuration memory 143 can be provided to retain personalization information for the capsule. At manufacture, a unique serial number and various hardware/software configuration parameters can be loaded. These parameters can be read by the programmable control processor 141 as often as necessary for proper operation of the capsule. The unique serial number can be used to identify the capsule to an associated data receiver system to facilitate correlation of test results to patients. Alternatively, a unique serial number or other identifier can be associated with the capsule by other methods, such as by a magnetic or optical tag or indicia, to correlate the capsule and test results to a particular patient.

The power control module 145 is used to manage power to some or all portions of the capsule. The power control module 145 can be used to conserve battery power through various load management schemes including, but not limited, to activating and deactivating various electrical modules within the capsule. The communication link module 146 accepts digital data words from the programmable control processor and formats them for correct transmission via the transmitter 122.

Referring once again to FIG. 2, the capsule 100 can include a power connection means 150. In one embodiment, the power connection means is a magnetic reed switch that is in series with the battery 110 and the remainder of the capsule electronics modules. Alternatively, active switches such as one based on a Hall-effect sensor can be applied. Choice of switch means is based on current carrying capacity and shelf life requirements.

In operation, the power connection means 150 can be “open” or in the disconnected state when an appropriately poled magnetic field is placed in proximity to the switch. When the magnetic field is removed from the proximity of the switch or an opposing field is provided to cancel the first field, the power connection means 150 can be “closed” or in the connected state, such that the capsule is operational.

Referring now to FIG. 6, the capsule can be enclosed in a protective package 160. The protective package 160 provides protection from physical abuse and from various environmental contaminants (e.g. dust, moisture, and bacteria). According to one embodiment, a magnet can be included in the protective package 160; wherein the magnet is appropriately poled and positioned to maintain the power connection means 150 in the “open” state when the capsule is contained within the protective package 160. When the patient removes the capsule from the protective package 160 prior to ingestion, the power connection means 150 is released to the “closed” state and the capsule electronics is activated. As shown in the figure, a magnetic structure 161 can be associated with one of two package parts 160A/160B such that when the package parts are separated to open the package and remove the capsule, the power connection means 150 is released to the closed state. Alternatively, other methods of activating capsule power can be used, including without limitation mechanical activation (such as with mechanical switches or materials that are moved, removed, or articulated when the package is opened), light or optical activation, vacuum or air pressure activation, and the like.

Radiation Detecting Capsule

FIGS. 2 and 3 show an embodiment of the detection capsule employing radiation detection. This capsule can be used with a radiolabeled differentiator. As the capsule travels along the GIT, the detector is brought into close proximity to tissues of the esophagus, stomach, small bowel, colon and rectum. This proximity can provide improved sensitivity and specificity compared to traditional external gamma radiation detection and imaging means such as Gamma Cameras and SPECT imagers and allow for the detection of small pre-cancerous and cancerous lesions that might otherwise escape detection. The detector will also sense signals coming from anatomical structures near by, including the pancreas, kidneys, spleen, bile ducts, gallbladder, liver and the genitourinary system, in addition to circulating marker material not yet bound to cancerous tissue. It can be desirable to choose the isotope, the detected energy range, to assist in suppression of these signals, or use other methods to suppress or account for these signals.

The capsule can include a detector module 130 comprising a suitable detector 132, a preamplifier 131, and a pulse-shaping amplifier 133. The detector is preferably a solid-state radiation detector. The detector module 130 is provided to have adequate dynamic response to allow unambiguous collection of high and low count-rate decay events. High count-rate decay events arise from unbound markers circulating in the patient's blood pool and temporarily resident in various non-cancerous tissues as a result thereof. Low count-rate decay events arise from the plurality of cancerous tissue source. A count rate differential in excess of 1000:1 between high and low count conditions may be encountered.

Solid-state radiation detection devices and methodologies are preferred in one embodiment of the present invention. Alternatively, detector 132 can be a solid-state scintillation detector comprised of a solid-state photo-detector (such as the Detection Technologies PDB or PDC series) coupled to a scintillation crystal to convert the decay particle to a number of photons. A lower count threshold can be representative of a 1-50 nano-Curie source and the detector module 130 can be adapted to accommodate this level of activity.

The preamplifier 131 can be used to convert charges created in direct solid-state detection devices or current generated in the photo-diode of a scintillation detection device into a voltage output. The output voltage magnitude is proportional to the energy of the particle incident on the detector 132. The pulse shape of the output can be determined by various circuit elements.

Pulse shaping amplifier 133 accepts the output of charge preamplifier 131 converting it to an output voltage pulse. The amplitude of the output pulse can be linearly related to the magnitude of the input signal. The pulse shape can be substantially Gaussian with a predefined and constant width “w” and a variable height “h” depending on the incident energy of the particles impacting the detector.

The capsule can include a detector electronics module 140. The detector electronics module 140 can include detector support electronics and a control processor. In one embodiment, an Application Specific Integrated Circuit (ASIC) that contains a programmable control processor 141, a clock generation and timing module 142, a write-once configuration memory 143, a plurality of single channel analyzer (SCA) modules 144, a power control module 145 and a communication link module 146 can be employed.

At least one SCA 144 can be provided, and in one embodiment a plurality of SCAs 144 is provided to interpret the output of the pulse-shaping amplifier 133. A Single Channel Analyzer can be used to qualify the pulses provided to its input according to their amplitude, providing a pulse of constant (standardized) width and amplitude only when the input pulse amplitude falls within a specified range. A plurality of SCAs, set for contiguous amplitude ranges, is frequently referred to as a multichannel analyzer (MCA). It provides a histogram of the energy distribution of the particles interacting with the detector. Such analyzers can be constructed in a number of ways well known in the field of nuclear instrumentation. A plurality of SCAs can also be set for arbitrary, noncontiguous, non-overlapping or overlapping ranges, in which case they are not typically considered an MCA. Such an array of SCAs can be employed to register selected energy regions associated with the expected energies of incident particles from the radioisotope or radioisotopes employed.

Directors suitable for this application include direct detectors (DD) and scintillation detectors (SD). Direct detectors respond “directly” to incident particles: that is, the particles interact with the detector material, generating charge carriers. In solid-state detectors, these carriers are holes and electrons. The system then senses these charge carriers through current or voltage measurement. Scintillation Detectors have a different conversion mechanism. Typically, the incident particle interacts with a scintillation medium to cause a burst of light. This light travels out of the scintillation medium and into a photodetector. In the photodetector, the light interacts with the material to generate charge carriers, which are sensed by the system through current or voltage measurement. A suitable SD device can be a combination of a CsI:Tl scintillation crystal tightly coupled to a high efficiency photo-diode(e.g. the Detection Technology PDB series).

Directionality

Detectors can exhibit varying degrees of directionality: that is, a dependence on the sensitivity with direction of arrival of the incident particles. This directionality may be advantageous or disadvantageous. A shield can be used to provide additional directionality to a detector response curve. For gamma radiation, shields can be made from a high-Z (atomic mass) material such as lead or tungsten. A shield typically blocks radiation from a large region of space. It is usually characterized by its angular or dimensional extent. A collimator can be used to provide additional directionality to a detector response curve. For gamma radiation, collimators can be made from a high-Z (atomic mass) material such as lead or tungsten. Collimators are characterized by a large l/w (length/width (or diameter)) ratio in at least one plane. For simple calculations, the effect of a collimator is to eliminate all radiation that attempts to strike the detector at an angle greater than the acceptance angle of the collimator. In another view, the effect is to accept only those within the acceptance angle.

Phased Arrays

The signals from more than one detector can be combined to give directionality significantly different from that of the individual detectors. When an array of antennae is assembled, and their outputs are combined, they are typically referred to as a phased array. The approach is not common in nuclear detectors, but there are enough similarities that the term is used analogously herein.

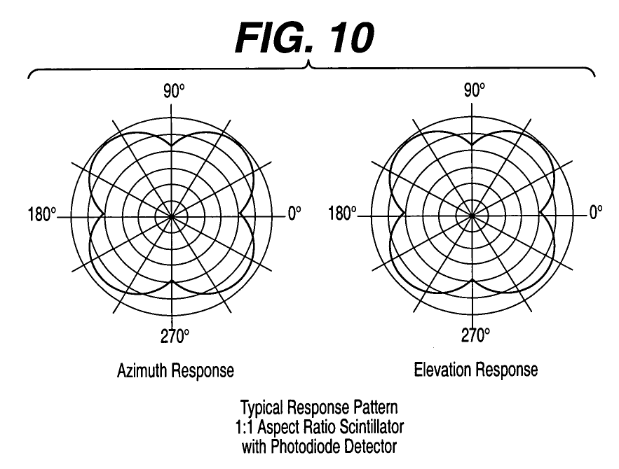

Without being limited by theory, for gamma particles of energies appropriate to this application, direct detectors can exhibit moderate directionality, and SD's nearly omni-directional responses. An example is shown in FIG. 10 where the scintillation crystal is a cube.

Use of radiation detectors can result in the radiation component from all sources (including tumor, circulating blood with marker material, organs filled with blood or otherwise containing marker material) being detected. Without being limited by theory, it is believed that the amount of marker material concentrated at small tumor can be orders of magnitude smaller than that resident in nearby organs. A detection approach that responds only to the smaller concentration, or which otherwise can discriminate between a tumor and other sources of radiation, would be advantageous.

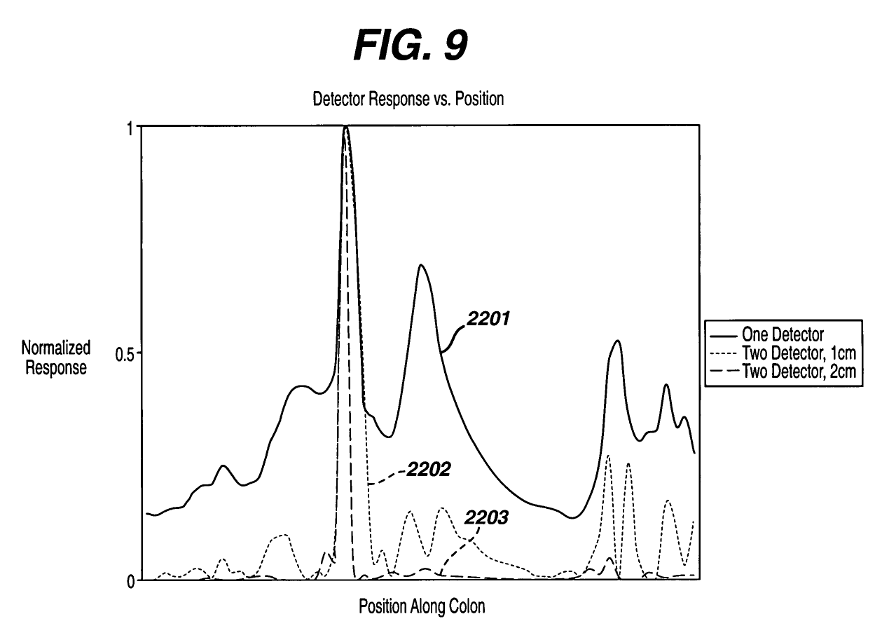

While collimators may be used to help in locating tumors, collimators occupy space on the capsule, and may have other disadvantages. According to one embodiment of the present invention, a capsule 100 can employ a detector array comprising at least two detectors. In such an embodiment, the first and second detectors can be disposed at opposite ends of the capsule 100. Each detector may or may not have associated with it a collimator device. The collimators can be used to restrict the solid angle through which the detector can sense incoming gamma particles. FIG. 9 shows a simulated response of a capsule bearing a single detector (curve 2201) and a two-detector system with two inter-detector spacings (1 cm, curve 2202, and 2 cm, curve 2203) as it transits a simulated GIT. In this example, the system response for the two-detector capsules is derived by taking the difference of the responses of the two detectors from each sampling period. This particular combination of the two responses is believed capable of providing a directional response that is largely insensitive to broad background sources. Other combinations of multiple detector responses (e.g. addition, multiplication, integration, differentiation) are also possible.

Magnetic Detecting Capsule

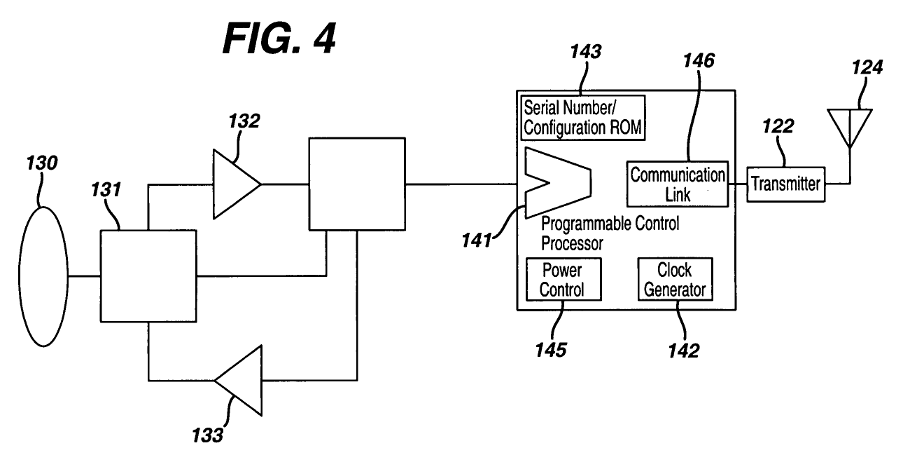

FIG. 4 illustrates components of a detection capsule employing magnetic detection which can be used with a magnetically labeled differentiator. Like the radiation approach described earlier, in transiting the GIT the capsule will be brought into close proximity with pre-cancerous and cancerous lesions. A magnetic detection device can be provided to respond to dipole and higher moment fields, which decrease with distance more rapidly than static (inverse-square) fields, providing increased rejection of signal which may result from circulating (e.g. in the blood stream or organs) marker material not yet bound to such lesions.

The capsule can include a coil 130, a transmit/receive switch 131, a detection amplifier chain 132, a stimulus amplifier 133, a signal conditioning and control block 134, and a processing and communications block similar to that described previously for a radiation-detecting approach, including a serial number/configuration ROM 143, a programmable control processor 141, a power control section 145, a clock generator 142, a message formatter 146, a transmitter 122 and an antenna 124.

In one magnetic detection approach, a magnetic field is briefly generated, utilizing the signal conditioning and control block 134 to construct a signal, which is amplified by the stimulus amplifier 133 and routed to the coil 130 by the transmit./receive switch 131. This results in either physical rotation of magnetic nanoparticles in the vicinity or rotation of their magnetic domains into varying degrees of alignment with their local field.

Next, the signal conditioning and control block 134 terminates the magnetizing signal and switches the coil 130 to connect to the detection amplifier chain 132. There now being no an aligning field, the orientation of the particles or their magnetic domains return to a random state. This change depends upon a number of factors, among which is the temperature, the size of the particles, and various parameters of the magnetic material itself. For a given situation, however, there is typically a characteristic “relaxation time” associated with the process.

While the particles or domains are returning to random orientations, their motion results in a detectable signal, of bandwidth which can be approximately the inverse of the relaxation time. The detection amplifier chain 132 can incorporate low noise amplifiers and filters to set its bandwidth to an appropriate value to pass these signals while substantially rejecting man-made and natural electromagnetic interference. Further processing, in the form of temporal qualification or pattern matching, may be applied to increase sensitivity or interference rejection. Conversion of the received signals or representative parameters into a digital form suitable for temporary storage and assembly into messages to be transmitted by the processing and communications block can also be incorporated. While the method just described contemplates both the stimulus and response equipment to be located within a capsule, it will be apparent that power or other constraints may require one or the other to be located outside the patient's body.

Position Tracking

During the course of travel through the GIT, the capsule may experience forward motion, retrograde motion, and tumbling. Accordingly, it may be desirable to provide a device for determining and/or tracking the position of the capsule in the GI tract. For instance, inertial, electrical, electromagnetic, magnetic, ultrasonic, and physical measurements can be employed track free or constrained body motion. For instance, single or multi axis accelerometers can be employed to determine position of the capsule 100. In the application of colon cancer screening, the usual action to be taken following an indication of high probability of cancerous tissue would be a thorough visual examination of the entire lumen via colonoscopy. While precise measurement of the capsule's position along the tract is not essential, an approximate confirmation of position would be useful for the diagnosis, as well as potentially to improve the integrity of the detection.

One aspect of the present invention is a method for position tracking. In a radiation-based application, small amounts of radioisotope would be placed at anatomically significant locations. These radioisotopes would preferably be chosen for a long half-life and existing availability, such as cobalt-57, commonly used for check sources. Suitable external locations would be established by external anatomy, palpation, or other means. Examples include the base of the sternum, roughly marking the start of the small intestine, and the crest of the right iliac bone, roughly marking the end of that organ. The isotopes would be contained in a durable enclosure and applied externally using a disposable adhesive patch designed to remain on the patient's skin for the duration of a typical test. When the PDCU is returned to the prescribing physician, the radioisotope packets would be returned and cleaned for re-use. A similar concept could be applied for magnetic detection systems, where a particular spatial pattern of responding material, or a temporal modulation of a local field or response attribute, could be detected by the capsule and either reported or filtered from the response data by the capsule.

During the capsule's transit of the GIT, the detectors and associated circuitry would be able to distinguish these external sources by their characteristic energy spectrum, it being different from that of isotopes used for marking. The latter can have a short half-life, and energies appropriate for moderate penetration, whereas the former can have longer half lives for economy and greater range for convenience. Observing the energy spectrum as a function of time allows the capsule, or a user examining the data, to more closely estimate the location of the capsule at any time. The number of external sources would be chosen depending upon the degree of localization accuracy required.

Orientation Tracking

Location tracking, described previously, provides information about the position of the capsule. Such location is specified by translations with respect to the origin of some coordinate system, and its measure is units of length. A full description of the capsule in space also involves its orientation. Such orientation is specified by rotations with respect to the axes of some coordinate system, and its measure is units of angle. Directional detectors, such as described herein, are useful for suppressing the signal contribution from large uniform distributions of radioactive marker. On the other hand, if the capsule tumbles (rotates about one or more axes), directional detectors can enhance the contribution from such distributions. Accordingly, in one embodiment of this invention, the capsule includes an orientation sensor. This may be implemented by one or more miniature electromechanical system (MEMS) angular rate sensors (e.g. measurement of angular velocity of capsule 100). The outputs from these sensors may be reported to the PDCU along with data from the radiation sensors, or utilized in the capsule to qualify or modify the radiation sensor data. For instance, if the output from such a rate sensor indicates the capsule is rotating such that the detector is “sweeping” past the liver, this information can be taken into account in interpreting the data from the detector.

Construction of the capsule can include the use of high density components. ASICs, hybrids, flexible and 3D circuits can be employed. In one embodiment, the capsule 100 can have a length of no more than about 1.5 inch, more particularly no more than about 1.0 inch, and a diameter or maximum width of no more than about 0.75 inch, more particularly no more than about 0.5 inch.

A bio-available compound can be included as an element of the capsule, such as a bioabsorbable coating. Depending on the application, a delayed release or immediate release coating can be applied over the coating on the exterior surface of the capsule to provide a desired release rate of the compound.

Endoscope Application

In an alternative embodiment, a detector can be employed with an endoscope. Position of the detector can be determined directly from graduations on the shaft of the endoscope. Furthermore, a detector can be employed with existing endoscopes which provide rotational constraints, and angulation controls that yield enough information about the orientation of the tip of the endoscope that no orientation sensing features are likely to be required in the detection mechanism. One embodiment of an endoscope-based device could take the form of a detector module that can be attached to the tip of an existing colonoscope, gastroscope or other flexible edoscope. The connecting wires could be secured to the outside of the scope or passed through a working or instrument channel normally provided in flexible endoscopes. In another embodiment, a detector module including a radiation or magnetic detector can be directed through the working or instrument channel of an existing flexible endoscope. The detecting module could be advanced beyond the working channel into the visualization field. It would then be possible to immediately inspect a region indicated as potentially cancerous, using the existing visualization features and capabilities of flexible endoscopes. Such simultaneous detection and inspection could be used as a follow up to results provided by a capsule-based detector.

Data Collection and Communication

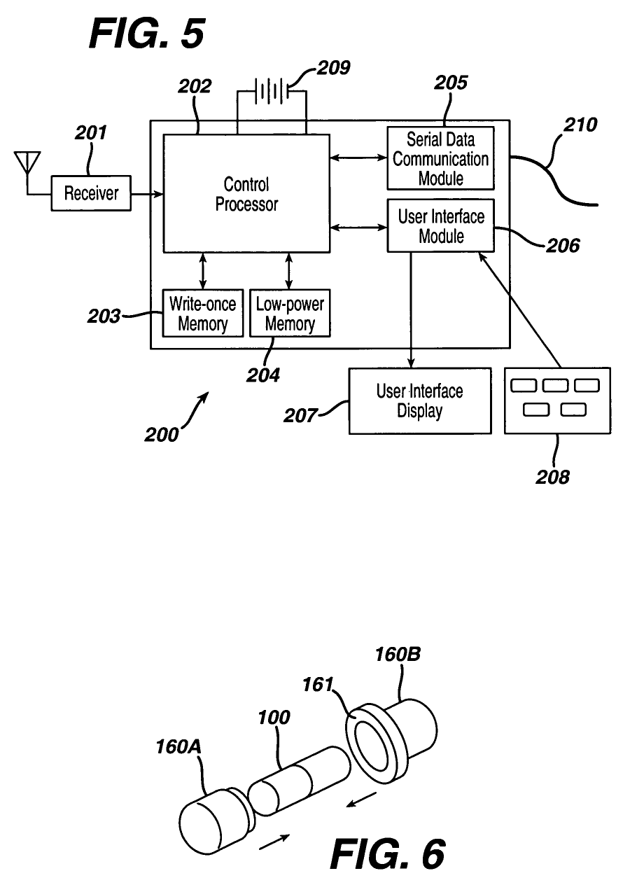

Referring now to FIG. 5, a patient data collection unit 200 (PDCU) for receiving data transmitted from the transmission module 120 can be employed to store data. The data collection unit can be attached to the patient (such as by clipping on to clothing) or be positioned in a room within receiving distance of the capsule 100 within the patient. The data collection unit can include a receiver 201, a control processor 202, a write-once memory 203 for storing configuration information and a unique serial number, a low power memory 204 for storing received data, a serial data communication module 205, a user interface module 206, a user interface display 207, a plurality of control buttons 208, and a battery 209. In one embodiment, the receiver 201, control processor 202, memories 203 and 204, communication module 205, and user interface module 206 can be combined within a single Application Specific Integrated Circuit (ASIC).

The receiver 201 can be selected to be compatible with the transmitter 120 and can convert radio signals to a digital data stream that is applied to the control processor 202. The control processor 202 can be based on a common commercial microcontroller core such as one based on the Intel 8051 8-bit processor instruction set and architecture. Instructions governing the operation of the data collection unit can be stored in the read-only memory embedded in the control processor core module. The microcontroller core can also provide for the management, control and data transfer between all portions of the ASIC and attached components. The write-once memory 203 can be used to store configuration information. Configuration information can be entered at the time of manufacturing or through connection to a physician workstation 400 shown in FIGS. 1 and 6. At the time of manufacture various parameters and a unique receiver unit serial number can be stored. When the receiver unit is activated at the physician workstation, other information such as a unique physician identifier code, the capsule serial number, activation date and time, patient number and name, and test type can be transferred to the data collection unit and stored in the write-once memory.

The low-power memory 204 can be used to store data delivered by the capsule. The memory can retain data during any low-power operation modes supported by the control processor and for up to for instance 2 hours when the battery 209 is removed for replacement. Information that can be stored in the low-power memory 204 for each message received from the capsule transmitter 120 can include: the time the message arrived, the complete content of the received message, and a series of data items to ensure data integrity. Such data integrity information can include data such as a Cyclic Redundancy Check (CRC) word and/or a multi-bit Error Correction Code (ECC).

The serial communication module 205 can be employed to connect the data collection unit to external computing and communications resources. In one embodiment, the module can contain a serial modem for connection to a telephone subscriber network or to the physician workstation. Alternatively, a USB connection, infrared communications or other standard computer interface can be supplied. To assure compatibility with the widest variety of telephone subscriber networks, the data communications rate can be selected to be as low as practicable with 9600 baud signaling considered being sufficient. However, higher or lower data communication rates can also be used. The user interface module 206 connects to the user interface display 207 and user control buttons 208 to the control processor 202. This module can perform any data formatting and device control operations required to efficiently display character and limited graphic information on the user interface display. It can also provide appropriate level translation and “de-bouncing” between the user control buttons 208 and the control processor 202.

The user interface display 207 can be used to present text information and graphics to the user. The display can be of the Liquid Crystal Display (LCD) type with or without backlighting. Various models of the data collection unit can be provided with various levels of graphic and information display sophistication. The user control buttons 208 can comprise a plurality of “push button” switches. In the preferred embodiment, the switches are all momentary single pole, single throw (SPST) type based on a pressure sensitive membrane switch technology. At least one button can be used to control the power state of the data collection unit. The battery 209 powering the data collection unit 200 can be relatively inexpensive, such as a 1.5 volt “AAA” battery.

At the conclusion of the testing period (i.e. after the capsule has passed through the patient's entire gastrointestinal tract) the data collected by the Patient Data Collection Unit 200 can be uploaded via an electronic connection, data line or over an internet connection to the Data Collection and Analysis Center 500 (FIG. 1), or the PDCU and its stored data can be delivered physically by postal services or common carrier to a desired location. The data can be transferred to the Data Collection and Analysis Center 500 directly by the patient (e.g. through an Internet connection or modem connection via a Personal Computer located in the home) or can be transferred by a remote collection and communication facility operated by an agent such as a pharmacy, clinic or physician's office.

Data Collection and Analysis Center

The Data Collection and Analysis Center 500 (DCAC) can comprise computing, communication, and operator interface resources. The DCAC can include one or more Internet Servers. The internet servers can have a plurality of modems connected to a plurality of telephone subscriber network assets. The internet servers can be dedicated to maintaining the database of capsule and data collection unit serial numbers, physician identification numbers and associated physician information, test performed tests analyzed and billing status. For diagnostic purposes, each internet server can be selectively connected to an operator interface unit composed of a plurality of display screens, a keyboard, and pointing device. When data is communicated to the DCAC, it can be processed with a series of data analysis techniques that are used to assess the time sequence of differentiator/marker outputs to identify suspicious data regions. Once analyzed, the capsule serial number is matched with a database of patients, physicians, capsule serial numbers, and procedure type to determine diagnostic report type and electronic address for delivery of electronic reports. If a database match is found, the report is finalized and delivered in a secure, encrypted fashion to the electronic address on record.

One form of analysis of the data received would be to examine the rate at which particles are detected at the capsule, in a single (or cumulatively in several) energy ranges. For isotopes and anatomies where the signal-to-background ratio is high, this may be sufficient. In some, it may be the case that the strong background from circulating and excreted marker material will make it difficult to distinguish the small increment of signal resulting from a tumor, even with the significant range advantage provided by the capsule's close approach to it. In gamma scintigraphy, methods have been disclosed for distinguishing particles arising from nearby and more distant sources, based on the differing attenuation and scattering with range as a function of energy (see, for example, Kaplan, Miyaoka et al, “Scatter and attenuation correction for 111 In based on energy spectrum fitting,” Med Phys 23(7) July 1966). By choosing a marker isotope with multiple decay energies (such as 111 In), and observing the ratio of detection events between a high and a low energy band, improved rejection of strong but distant background counts can be achieved. The received energy spectra can be compared or fitted to a mathematical model of the spectrum of the isotope used for detection. The model of the spectrum of the isotope can be modified to take into account passage of the detector through the body and/or location of the substance containing the isotope in an organ. For instance, a sample or test model of what the spectrum would “look like” if due to the isotope being detected in a blood filled organ can be compared against the actual measured energy spectra, and based on the comparison, a probability can be assigned to the likelihood that the actual measured energy spectra corresponds to a tumor. Also, the number of counts or particle energy levels received in different energy bands can be compared (such as by ratio) to determine or estimate the distance to the source, which can be used to estimate the likelihood/probability that a peak in a particular energy band corresponds to a tumor. Further improvement may be made through observation of a broad energy spectrum, whereby Bremstrahlung components can be rejected by mathematically fitting a trial distribution to the parts of the spectrum more distant from the emitter peaks, and subtracting those distributions from the raw data. Similarly, the broadening of the spectrum due to Compton scattering in the body and detector may be advantageously modeled and employed to correct the raw count data, improving the quality of the count ratio measure.

In addition to standard data transform methods such as Fourier transforms, it may be desirable to employ other transforms, such as the Hilbert or Hilbert-Huang transform. Such methods are characterized herein as “nonuniform sampling transforms.” Furthermore, multivariate analyses and multi-layer learning (“connection”) machines may be employed for discerning underlying patterns for which no higher level abstraction may be apparent. Such methods are characterized herein as “parametric transforms.”

Physician Workstation

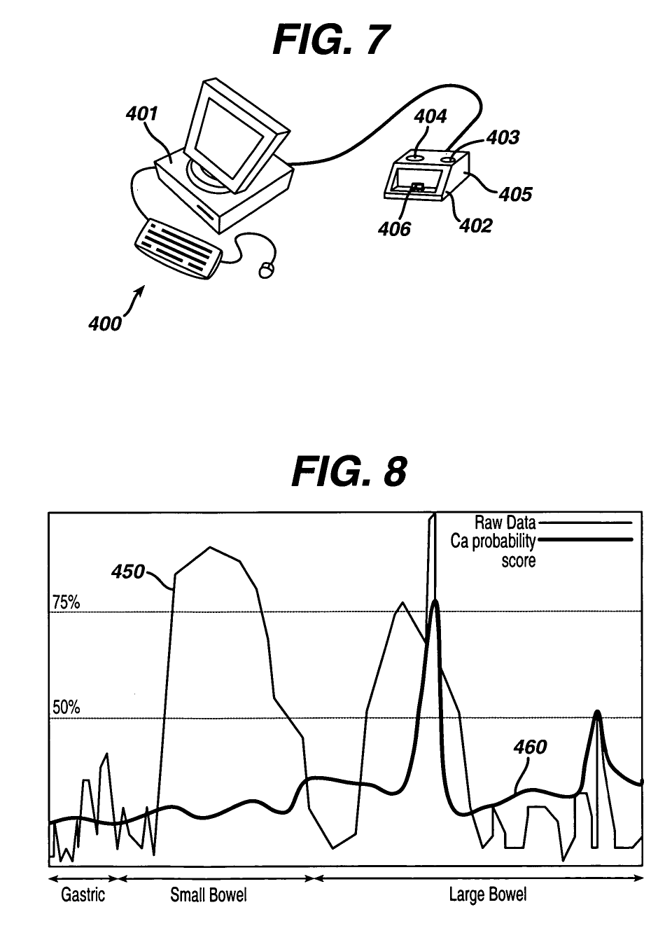

Referring to FIG. 7, a Physician Workstation and Analysis System 400 (PWAS) can also be employed. The PWAS can be based on a standard personal or office computer 401. A capsule interface unit 402 can be provided. For a radiolabeled MAb substance provided in vial (FIG. 1) , the capsule interface unit 402 can include a capsule receptacle 403 for receiving the capsule 100 enclosed in protective package 160; a vial receptacle 404 for receiving the vial containing the radiolabeled Mab substance (shown in FIG. 1); a built-in version of the patient data collection unit, the built-in data collection unit 405; and a socket 406 to accept the cable from or directly plug into a Patient Data Collection unit 200. The capsule interface unit 402 can also include an internal communication system such that all components (the capsule 100, marker vial 300, and Patient Data Collection Unit 200) can be secured in the correct sockets to download the data from the capsule interface unit 402 into the standard personal or office computer 401. The capsule interface unit 402 can further include one or more barcode readers. Barcode reader can be used to read one or more indicia (e.g. bar codes) containing information such as serial numbers associated with capsule 100, the vial, and/or Patient Data Collection Unit 200.

Computer 401, which can be a PC or MAC computer, a workstation computer, or a Palm Pilot or other personal data assistant (PDA), can include a connection port, a user interface (e.g. keyboard, mouse), and a monitor. The connection port, which helps connect capsule interface unit 402 to standard personal or office computer 401, can send and receive data to and from capsule 100, the vial, and/or Patient Data Collection Unit 200 via capsule interface unit 402. The data sent to computer 401 can be encrypted for security measures. Computer 401 can employ any suitable operating system. Computer 401 can further include software for use in analyzing data received from unit 402 and/or PDCU 200. The software program can further include a decryption code used to decode any encrypted data sent from the capsule interface unit 402.

The capsule interface unit 402 can be connected to the computer 401 via any one of a number of standard computer peripheral methods such as, but not limited to; an RS232 serial interface, an IEEE1394 or USB interface, via an Ethernet cable or phone line over the Internet or a Local Area Network, a parallel printer-like data interface, a fiber optic interface, a custom PCI card interface, or an infrared or RF interface. The software in the computer 401 can also be used to facilitate operation of the capsule interface unit 402.

Functions that can be provided by the PWAS 400 include but are not necessarily limited to 1) verify the operability of the capsule 100; 2) verify the operability of the Patient Data Collection Unit 200; 3) verify the activity level of the differentiator (such as a radiolabeled MAb embodiment); 4) program patient, physician and test type information into the Patient Data Collection Unit 200; 5) communicate, via a secure, encrypted data method, with the Central Processing Center 500 the name and ID of the physician and patient, the serial numbers of the capsule 100 and the Patient Data Collection Unit 200, type of test requested and administered, and time of injection of substance 300.

It can be a further function of the physician workstation to receive encrypted secure data report from the Data Collection and Analysis Center 500 and subsequently display or print that report on demand. To acquire the several pieces of data to be entered by the physician or an associate, a modern user interface, such as a graphical user interface, can be provided for operation on the standard personal or office computer 401.

To activate and/or verify operability of the capsule 100, the capsule interface unit 402 socket or port that is adapted to accept the capsule complete with its protective package 160 can include an activation mechanism, such as a magnetic means (assuming that the capsule power is magnetically activated) to override the field created by the magnet contained in the protective package. The built-in data collection unit 405 can be adapted to receive and/or respond to data provided by or stored in the capsule 100 and provide that data to the computer 401 for performing basic data validation checking.

To verify operability of the patient's data collection unit 200, it can be connected to the workstation capsule interface unit 402 via the data collection unit interface cable 210 (FIG. 4). With the capsule 100 transmitting data, the output from the patient data collection unit 200 can be compared with the output from the built-in data collection unit 405. To verify the activity level of the differentiator (radio-labeled MAb) substance 300, the vial containing the substance 300 can be inserted into the socket provided in the capsule interface unit 402. With the capsule 100 also inserted in its mechanical socket, the radioactive count levels received from the vial can be transmitted to the built-in data collection unit 405 and the patient data collection unit 200. The information can then be communicated to the computer 401 to be checked against a range of acceptable values.

After verifying correct operation of the various system components (i.e. capsule 100, patient data collection unit 200 and the differentiator substance 300 in the vial), physician entered data and various calibration and configuration codes determined by the software plus patient information can be transmitted to the patient data collection unit 200 via the data collection unit interface cable 210. Within the patient data collection unit 200 this data can be stored in an appropriate location within the write-once memory 203.

FIG. 8 shows a report format that can be displayed in written or electronic form at the PWAS 400. On this report, the raw data corresponding to radiation counts per unit time received by the detector is normalized and presented as raw data curve 450 with respect to the approximate location in the GI tract indicated on the horizontal axis. As a result of data processing that takes place at the Data Collection and Analysis Center 500, a predictive score can be provided (such as is depicted as Ca Probability Score curve 460 in FIG. 8, depicting the probability (likelihood) that a concentration of marker has formed at a position along the gastrointestinal tract). The importance of the predictive score can be determined by clinical reports and the experience of the physician analyzing the results. In general, the purpose of the predictive score can be to indicate if a peak in the raw data curve 450 indicates cancer or background radiation such as from the material provided in the marker vial 300 stored in the liver or spleen. For instance, in FIG. 7, the peak in the raw data curve 450 corresponding to the small bowel is not likely to indicate the presence of cancer in the small bowel due to the probability value provided by the Ca Probability Score curve 460 corresponding to the small bowel.

Two Differentiator Method

In a different embodiment, two or more differentiator agents can be used in order to increase the accuracy of the test. The accuracy of a single differentiator such as a monoclonal antibody can be limited by its distribution to healthy organs as well as disease areas. For example, monoclonal antibodies tend to distribute to the liver, kidneys, spleen, urinary bladder and bone marrow. This can give rise to false positive readings, or reduced specificity, since signals emitting from one of those organs are falsely interpreted as emanating from disease. Moreover, the radioactivity coming from the circulating portion of the injected MAb may be much higher than that emanating from a small tumor or lesion, thus masking the real diseased tissue. The physician is then unsure as to the nature of the signal: is it emanating from diseased cells, or does it merely represent normal distribution of the antibody throughout the body?

Rather then only receiving one differentiator, for example a radiolabeled MAb specific to disease, the patient also receives a second MAb, albeit one which is marked by another particle. For example, if the original drug were a MAb marked with radioactive material such as ^{99m}Tc, then the co-administered agent could be a similar MAb marked with a different radioactive label, such as ¹¹¹In. Moreover, the second agent could be designed so as to concentrate in similar concentrations in the different body compartments (e.g. kidney, liver, blood, and liver). To this end, the second agent could have similar molecular weight, charge and physical characteristics, but would have a different binding surface. A practical way to achieve this could be to use two monoclonal antibodies of the IgG type, one with specificity to the tumor marked with ^{99m}Tc, the other being a nonspecific IgG antibody marked with a different radioactive marker such as ¹¹¹In.

Upon administration to the patient, both MAb's can concentrate in generally equal amounts within the body compartments. However, there will also be some tumor uptake of the MAb that is designed to attach to the tumor. Using a radioactivity analyzer (e.g. multi-channel spectral analyzer) that can differentiate between the isotopes, one can determine for each area of the body how much radioactivity is emanating from each of the two labels. Since the labels are designed or chosen so as to have similar molecular weight and composition, they can be very similar in their pharmacokinetic and pharmacodynamic qualities. Thus, by appropriately scaling and/or subtracting the radioactivity intensity emanating from one source from that coming from the other one should get a negligible reading of radioactivity. This will generally be the case, except where there is a tumor to which one of the antibody types attaches, in which case this MAb will have stronger binding and the radioactivity emission from this area will be markedly higher than that coming from the isotope attached to the second antibody. The final response to the physician can be the net result of subtracting the two radioactivity levels, which may significantly reduce confusion associated from background interference, or the non-specific distribution explained above.

By way of prophetic example, a method can include the following steps:

(1) providing a specific differentiator for a tumor or another abnormal tissue such as inflammatory or necrotic tissue. Possible differentiators include but are not limited to a monoclonal antibody, peptide, nucleic acid (nucleotide), nanoparticle, or other.

(2) providing a marker material that is bound to the differentiator or that binds to it upon administration to the patient. Possible materials include but are not limited to radioactive nuclides such as ^{99m}Tc, fluorescent molecules such as one of the porphyrin family of chemicals, ultrasonic contrast agents or other.

(3) providing a material similar to the material in step 1, for example a protein of similar molecular weight, charge and 3-D structure. This agent is different from that in step 1 in that it does not attach to the same moiety in the body. To illustrate, if a MAb from the IgG immunoglobulin class is chosen in step 1, such as the commercial drug Oncoscint, a suitable material to choose as the second agent (3) would be a IgG antibody that is not specific to a known moiety in the body. Alternatively, one can use or a mixture of nonspecific IgG. Finally, one can choose an IgG whose Fc portion or antigen recognition area does not fit a specific receptor. For example, an IgG antibody whose Fc portion consists of a repetitive sequence of one amino acid, such as Alanine.

(4) providing a marker material bound to the agent in (3), which is different from that in step 2. For example, if the radioactive isotope ^{99m}Tc was provided in step 2 above, then the isotope ¹¹¹In can be chosen here.

(5) providing a detector system that detects the signals emitted by markers (2) and (4), be it a radioactivity detector, magnetic field sensor, or other signal. The system should be able to differentiate between the two different sources. For example, radioactivity resulting from the presence of ^{99m}Tc should be differentiated from that resulting from ¹¹¹In due to the widely separated decay energy of the respective gamma radiation.

(6) Scaling and subtracting the signals coming from the two markers or otherwise processed to provide a result which can be exhibited to the physician.

The method may also allow the user to increase the level of differentiator given to patient in order to increase its sensitivity, without concern for background increasing noise. Thus, the system can increase both sensitivity (e.g. what proportion of patients are diagnosed) and specificity (given a positive result, what is the likelihood that that patient is indeed sick).

Avidin/Biotin Method

In another embodiment, in order to increase test accuracy one may use materials that strongly bind to each other, but have less binding affinity or none at all to other chemical moieties. Apart from antibodies mentioned above, other materials that have relatively high binding affinity to each other can be used. In nature, or when mixed together under laboratory conditions, such agents will strongly bind to each other in a tight, nearly permanent fashion.

One example of these couples is the Avidin-Biotin couple. Biotin is a vitamin from the B complex. It is a colorless crystalline vitamin with chemical composition C₁₀—H₁₆—N₂—O₃—S. It is essential for the activity of many enzyme systems. Avidin is a protein found in uncooked egg white that binds to and inactivates biotin.

Biotin's and Avidin's attraction to each other is often used in laboratory experiments, often for diagnostics. The relationship between Avidin and Biotin has also been used by the pharmaceutical industry in order to develop guiding mechanisms for drugs. See Karacay H, et al. Development of a streptavidin-anti-carcinoembryonic antigen antibody, radiolabeled biotin pretargeting method for radioimmunotherapy of colorectal cancer. Reagent development. Bioconjug Chem 1997 July-August;8(4):585-94, and Schultz A. Tetravalent single-chain antibody-streptavidin fusion protein for pretargeted lymphoma therapy. Cancer Res 2000 Dec. 1;60(23):6663-9 which are incorporated herein by reference.

Other proteins with similar structure as Avidin or derivatives thereof may be used in order to optimize its binding, reduce clearance, improve its pharmacokinetic or pharmacodynamic attributes or induce other favorable effects. For example, Recombinant Streptavidin (rSAv) may be used instead of Avidin. Furthermore, it may be desirable to modify rSAv in order to get a more favorable action, for example by reducing its rather high kidney localization. Methods that have been described in the medical literature to that end include succynilation of rSAv using Succinic Anhydride. See Comparison of Biotin Binding and Tissue Localization of 1,2-Cyclohexanedione and Succinic Anhydride Modified Recombinant Streptavidin,. Bioconjug Chem 2002 May-June; 13(3):611-20; Evaluation of Methods for Decreasing Localization of Streptavidin to Kidney while Retaining its Tumor Binding Capacity, Bioconjug Chem 1998 May-June;9(3):322-30],which are incorporated herein by reference.

In one embodiment, a method can be used to employ the association between Biotin and Avidin or other similar “couples” in order to increase the accuracy of capsule-based cancer diagnosis. By way of prophetic example, the method can include the following steps:

(1) Providing a patient with a MAb or FAb or another differentiating molecule specific to disease such as cancer. Attached to the MAb is Avidin or Streptavidin, or another member of the Avidin family. Attachment of the Avidin or Avidin-like moiety to the MAb or FAb or other agent used as the differentiator may be achieved by genetic engineering creating a fusion protein as described by Schultz A. Tetravalent single-chain antibody-streptavidin fusion protein for pretargeted lymphoma therapy,. Cancer Res 2000 Dec. 1 ;60(23):6663-9, incorporated herein by reference.

(2) Allowing the drug to accumulate in diseased tissue, then giving the patient a clearing agent containing biotin or another molecule with very high affinity to the initial agent. Biotin binds strongly to the drug given in step 1 and is still free in the body. Thus, any remaining drug is that which is bound to the specific target. Alternatively, in another embodiment one may wait ample time for the drug given in step 1 to naturally clear from the body.

(3) providing to the patient a biotin attached to a radioactive or other marker such as ^{99m}Tc, a magnetic particle, a fluorescent marker, or other marker. The Biotin binds the Avidin and marks the disease with radioactivity or another signal providing mode, depending on the marking agent attached to Biotin.

(4) administering the swallowable capsule with detector to the patient before, during or after the above procedure.

Operation

The following operational description refers to devices and methods of the present invention wherein a cell marker substance comprising a radiolabeled monoclonal antibody is employed. For purposes of screening a target population for colon cancer in a relatively non-invasive procedure, the following operational steps can be employed.

A patient requiring screening can present to a physician or physician associate for a colorectal cancer screening test. In the implementation employing radiopharmaceuticals, the physician or related staff can order and receive a screening kit from a pharmacy licensed to dispense nuclear medicine materials and taken delivery of that test kit earlier on the date of the patient visit. In the implementation employing magnetic detection, the materials are presumably not regulated and can be drawn from local stock.

Upon arrival of the patient, the physician can place components of the kit in a special fixture at the PWAS 400. The components of the kit can include a swallowable detection capsule 100, a patient data collection unit (PDCU) 200, and an injectable cell marker substance 300 (CM) provided in a vial. The PWAS 400 and associated software can be used to verify the operability of all of the kit components and program certain information into the PDCU 200.

Once the kit is determined to be operable, the physician can inject the cell marker substance 300 into the patient and the patient can be instructed to swallow the detection capsule 100. The patient can be instructed on the use of the PDCU 200 and it can be attached to the patient in the same fashion as a pager, cell phone or wrist watch. Alternatively, the patient may be instructed to wait an optimum time before swallowing the capsule, such delay possibly acting to improve the test results by allowing a certain degree of natural elimination of circulating cell marker material (CM).

At this point, the patient returns to normal daily activity as the capsule 100 and detector travel through the GI tract from the esophagus through the stomach, small intestine, colon (large intestine) and eventually is expelled through the anus with stool during a bowel movement.

As the detector travels through GI tract, it is periodically measuring and reporting the signals emitted from various sources in the patient, or parameters (e.g. voltages) representative of those signals. This information can be combined with a unique identifier code for the capsule 100 and a timing indication as it is transferred to the PDCU 200. The PDCU 200 can be used to collect and store all of the information from the capsule 100 for subsequent communication to the Data Collection and Analysis Center (DCAC) 500.

Once the data arrives at the Data Collection and Analysis Center 500, a series of analytical routines can be applied to the raw data and a procedure specific report can be generated. That report can be routed to the physician (such as to the PWAS 400) and can include information that verifies operability of the kit and encodes the patient and physician information into the PDCU 200.

It will be recognized that equivalent structures may be substituted for the structures illustrated and described herein and that the described embodiment of the invention is not the only structure that may be employed to implement the claimed invention. In addition, it should be understood that every structure described above has a function and such structure can be referred to as a means for performing that function.

While preferred embodiments of the present invention have been shown and described herein, it will be obvious to those skilled in the art that such embodiments are provided by way of example only. Numerous variations, changes, and substitutions will now occur to those skilled in the art without departing from the invention. Accordingly, it is intended that the invention be limited only by the spirit and scope of the appended claims.

What is claimed:

1. A swallowable capsule comprising:
a detector;
a pulse shaping device; and
at least one single channel analyzer.

2. The capsule of claim 1 comprising at least two detectors.

3. The capsule of claim 1 wherein the detector is a radiation detector.

4. The capsule of claim 1 wherein the detector detects magnetic material.

5. The capsule of claim 1 comprising a plurality of single channel analyzers.

6. The capsule of claim 1 comprising a multiple channel analyzer.

7. The capsule of claim 1 wherein the capsule is coated with a material.

8. The capsule of claim 1 wherein the capsule is coated with a material for modifying the capsule's transit through the GIT.

9. The capsule of claim 1 wherein the capsule includes a magnetically-activated switch.

10. The capsule of claim 1 wherein the capsule includes an angular rate sensor.

11. A system for detecting particular tissues, the system comprising:
a capsule comprising a detector;
a substance for associating with the particular tissue, wherein the substance is capable of being detected by the detector; and
a machine for verifying at least one of the detector and substance are suitable for use.

12. A method for detecting target cells in a patient comprising:
marking target cells in the patient with a substance capable of being detected;
directing a detector through a naturally occurring body lumen in the patient to
detect signals from the substance; and
mathematically transforming data representing at least some of the signals detected.

13. The method of claim 12 comprising the step of verifying at least one of the amount, concentration, and activity of the marking substance.

14. The method of claim 12 wherein the substance comprises a monoclonal antibody.

15. The method of claim 12 wherein the substance comprises a peptide.

16. The method of claim 12 wherein the substance comprises a nanoparticle.

17. The method of claim 12 wherein the substance comprises a nucleotide sequence such as mRNA or DNA corresponding to a genetic material monoclonal antibody.

18. The method of claim 12 wherein the substance comprises a liposome or liposome structure.

19. The method of claim 12 comprising administering multiple radioisotopes to a patient.

20. The method of claim 12 comprising acquiring energy spectra.

21. The method of claim 12 comprising fitting particle energy spectra to a model.

22. The method of claim 12 comprising fitting particle energy spectra to a model of the spectrum of an isotope.

23. The method of claim 12 comprising comparing received particle energies in different energy bands.

24. The method of claim 12 comprising employing multiple detectors.

25. The method of claim 12 comprising combining or comparing the outputs of multiple detectors to provide a spatial response pattern.

26. The method of claim 12 comprising comparing temporal variation of acquired data with predetermined patterns.

27. The method of claim 12 comprising employing multiple radiation sources external of a patient.
